(19) United States
(12) Patent Application Publication
(10) Pub. No.: US 2025/0372208 A1
(43) Pub. Date: Dec. 4, 2025

(54) Synthetic Augmentation of Multiple Sequence Alignment of Protein-Protein Interactions

(71) Applicant: A-Alpha Bio, Inc., Seattle, WA (US)

(72) Inventor: David NOBLE, Seattle, WA (US)

(21) Appl. No.: 19/224,703

(22) Filed: May 30, 2025

Related U.S. Application Data
(60) Provisional application No. 63/654,840, filed on May 31, 2024.

(51) Int. Cl. G16B 35/00; G16B 15/30; G16B 40/20

(52) U.S. Cl. CPC G16B 35/00; G16B 15/30; G16B 40/20

(57) ABSTRACT

The present disclosure provides a method of predicting a structure of an interface between a target peptide and a targeting peptide. The method leverages test pairs of variants of a target peptide and variants of a targeting peptide and their binding affinities measured by a high-throughput analysis. Synergistic pairs among the test pairs are selected and multiple sequence alignment (MSA) of the selected pairs is performed to predict a structure of the protein complex formed with the target peptide and the targeting peptide. Structure prediction using MSA of the synergistic pairs provides for improved results, thereby paving the path for downstream analyses, e.g., small molecule design for molecular glues or antibody design.

### CROSS-REFERENCE TO RELATED APPLICATIONS

The present application claims the benefit of and priority to U.S. Provisional Application No. 63/654,840 filed on May 31, 2024, which is incorporated by reference in its entirety.

### BACKGROUND

Protein-protein interactions play a crucial role in cellular biology, underpinning virtually all biological processes within biological organisms. They encompass broad functions, from contributing to the structural integrity of cells to mediating signal transduction, enzyme activity, and gene regulation. The interconnected networks of these interactions serve as the backbone of cellular machinery, allowing for complex, coordinated responses to environmental stimuli. Without this dynamic interplay, cells would be unable to function properly, ultimately disrupting the balance of life processes.

Understanding protein-protein interactions can aid in developing molecular glues. These biomolecules aid in the activation and stabilization of protein complexes. The mechanism typically involves the “molecular glue” binding to one protein, instigating a conformational change that improves its interaction with a second protein. As such, improved understanding of protein-protein interactions holds immense potential in drug discovery and therapeutic applications, with the capability to modulate disease-associated protein interactions.

Understanding protein-protein interactions can also aid in antibody discovery. Improved understanding of antibody-antigen binding affinity can aid in the optimized design of antibodies towards specific antigens. Spectrum-wide, from vaccine development to targeted therapeutics, a nuanced knowledge of protein-protein interactions brings about a significant edge in antibody discovery and refinement.

### SUMMARY

The present disclosure provides a method of predicting a structure of an interface between a target peptide and a targeting peptide. The method leverages test pairs of variants of a target peptide and variants of a targeting peptide and their binding affinities measured by a high-throughput analysis. Synergistic pairs among the test pairs are selected and multiple sequence alignment (MSA) of the selected pairs is performed to predict a structure of the protein complex formed with the target peptide and the targeting peptide. Structure prediction using MSA of the synergistic pairs provides for improved results, thereby paving the path for downstream analyses, e.g., small molecule design for molecular glues or antibody design.

In some aspects, the techniques described herein relate to a method of generating a predicted structure of an interface between a target peptide and a targeting peptide, the method including: obtaining sequences of (i) a first library of variants of the target peptide and (ii) a second library of variants of the targeting peptide; generating a plurality of test pairs, wherein each test pair includes one target variant selected from the variants of the target peptides and one targeting variant selected from the variants of the targeting peptide; obtaining binding affinity data for each of the plurality of test pairs; selecting one or more pairs out of the test pairs as one or more synergistic pairs based on the binding affinity data; performing multiple sequence alignment with the sequences of target peptides and targeting peptides in the one or more synergistic pairs; and generating the predicted structure of the interface between the target peptide and the targeting peptide based on the multiple sequence alignment.

In some embodiments, the techniques described herein relate to a method, wherein the one or more synergistic pairs are selected from test pairs for being a synergistic mutation pair.

In some embodiments, the techniques described herein relate to a method, wherein the one or more synergistic pairs are selected from test pairs having a combinative effect on binding affinity compared to individual effects of the target variant and the targeting variants.

In some embodiments, the techniques described herein relate to a method, wherein the one or more synergistic pairs are selected from test pairs for having a binding affinity below a threshold.

In some embodiments, the techniques described herein relate to a method, wherein the binding affinity data is obtained by a high-throughput analysis of binding between the test pairs.

In some embodiments, the techniques described herein relate to a method, wherein the high-throughput analysis is performed by a method including: expressing variants of the target peptide in the first library and variants of the targeting peptide in the second library on surfaces of two separate haploid strains of yeasts; and measuring rates at which yeasts of two separate haploid strains fuse into diploids, thereby obtaining the binding affinity data.

In some embodiments, the techniques described herein relate to a method, wherein the high-throughput analysis is performed in the presence of a mediating ligand.

In some embodiments, the techniques described herein relate to a method, wherein the binding affinity data indicate binding affinity among the variants of the target peptide, the variants of the targeting peptide, and the mediating ligand.

In some embodiments, the techniques described herein relate to a method, wherein the predicted structure of the interface is a structure of the interface in the presence of the mediating ligand between the target peptide and the targeting peptide.

In some embodiments, the techniques described herein relate to a method, wherein the predicted structure of the interface further includes a structure of the mediating ligand.

In some embodiments, the techniques described herein relate to a method, further including: generating a structure of a mediating ligand or selecting a mediating ligand that can facilitate binding between the target peptide and the targeting peptide using the predicted structure of the interface between the target peptide and the targeting peptide.

In some embodiments, the techniques described herein relate to a method, further including: producing the mediating ligand.

In some embodiments, the techniques described herein relate to a method, further including: testing a binding affinity of the target peptide and the targeting peptide in the presence of the mediating ligand.

In some embodiments, the techniques described herein relate to a method, further including: modifying the mediating ligand or selecting an alternative ligand to improve binding to the target peptide and/or the targeting peptide.

In some embodiments, the techniques described herein relate to a method, wherein the targeting peptide is an antibody, and the target peptide is an antigen.

In some embodiments, the techniques described herein relate to a method, wherein the first library of variants of the target peptide includes one or more homologs of the target peptide and the second library of variants of the targeting peptide includes one or more homologs of the targeting peptide.

In some embodiments, the techniques described herein relate to a method, wherein the one or more homologs of the target peptide and/or the one or more homologs of the targeting peptide are generated by a generative machine-learning model.

In some embodiments, the techniques described herein relate to a method, wherein the plurality of test pairs includes one or more pairs of a homolog of the target peptide and a homolog of the targeting peptide.

In some embodiments, the techniques described herein relate to a method, further including: identifying a first set of amino acid residues of the targeting peptide contribute to binding to the target peptide based on the structure of the interface and a second set of amino acid residues of the targeting peptide that do not contribute to binding to the target peptide sequence.

In some embodiments, the techniques described herein relate to a method, further including: generating an investigative variant of the targeting peptide by modifying one or more amino acid residues of the second set of amino acid residues of the targeting peptide sequence; and receiving binding affinity data on the investigative variant of the targeting peptide and the target peptide.

In some embodiments, the techniques described herein relate to a method, further including: determining that the binding affinity data on the investigative variant of the targeting peptide sequence and the target peptide sequence is greater than a threshold; and producing the investigative variant of the targeting peptide.

In some embodiments, the techniques described herein relate to a method, wherein performing the multiple sequence alignment with the sequences of target peptides and targeting peptides in the one or more synergistic pairs includes: performing sequence alignment of sequences of the targeting peptide in the one or more synergistic pairs; and performing sequence alignment of sequences of the target peptide in the one or more synergistic pairs.

In some embodiments, the techniques described herein relate to a method, further including: identifying amino acid residues in the targeting peptide and amino acid residues in the target peptide that contribute to interaction between the targeting peptide and the target peptide based on the multiple sequence alignment, wherein generating the predicted structure of the interface between the target peptide and the targeting peptide is further based on the identified amino acid residues in the targeting peptide and the identified amino acid residues in the target peptide that contribute to interaction between the targeting peptide and the target peptide.

In some embodiments, the techniques described herein relate to a method, wherein in the step of generating the predicted structure of the interface between the target peptide and the targeting peptide, the identified amino acid residues in the targeting peptide and the identified amino acid residues in the target peptide are used as constraints.

In some embodiments, the techniques described herein relate to a method, wherein generating the predicted structure of the interface between the target peptide and the targeting peptide includes: applying a structure prediction model configured as a machine-learning model to the multiple sequence alignment to predict the structure of the interface.

In some embodiments, the techniques described herein relate to a method, wherein the structure prediction model is a machine-learning model developed using multiple sequence alignments of natural protein sequences for training or inference, optionally wherein the natural protein sequences include natural homologs of the targeting peptide and/or the target peptide.

In some embodiments, the techniques described herein relate to a method or claim 26, wherein the structure prediction model is configured to constrain structure prediction by establishing the amino acid residue residues in the targeting peptide and the amino acid residues in the target peptide as component to the binding interface of the targeting peptide and the target peptide.

In some embodiments, the techniques described herein relate to a method, further including providing a confidence evaluation associated with the predicted structure of the interface between the target peptide and the targeting peptide, optionally wherein the confidence evaluation is represented by a predicted aligned error (PAE) score.

In some embodiments, the techniques described herein relate to a method, further including: generating a digital representation of the structure of the binding interface between the target peptide and the targeting peptide.

In some embodiments, the techniques described herein relate to a method, generating a graphical user interface of the digital representation of the structure of the binding interface between the target peptide and the targeting peptide, wherein the graphical user interface is configured for display on a client device.

In some embodiments, the techniques described herein relate to a method, wherein generating the graphical user interface presenting the digital representation of the structure of the binding interface between the target peptide and the targeting peptide includes: tagging, in the digital representation, the amino acid residues in the targeting peptide sequence and the amino acid residues in the target peptide sequence that contribute to binding of the targeting peptide and the target peptide.

In some embodiments, the techniques described herein relate to a method, wherein the first library of variants of the target peptide or the second library of variants of the targeting peptide comprises over 100 variants.

In some embodiments, the techniques described herein relate to a method, wherein the plurality of test pairs comprises over 10,000 test pairs.

In some aspects, the techniques described herein relate to a non-transitory computer-readable storage medium storing instructions that, when executed by a computer processor, cause the computer processor to perform the method disclosed herein.

In some aspects, the techniques described herein relate to a system including: a computer processor; and the non-transitory computer-readable storage medium.

In some aspects, the techniques described herein relate to a non-transitory computer-readable storage medium storing a predicted structure.

In some aspects, the techniques described herein relate to a graphical user interface for displaying a predicted structure.

In some aspects, the techniques described herein relate to a method of synthetic augmentation of multiple sequence alignment and structure prediction, the method including: receiving, from a client device, a query including a target peptide sequence and a targeting peptide sequence; querying a database to obtain one or more homolog pairs of the target peptide sequence and the targeting peptide sequence; generating a plurality of variants of the target peptide sequence and a plurality of variants of the targeting peptide sequence; transmitting the plurality of variants of the target peptide sequence and the plurality of variants of the targeting peptide sequence for binding affinity assaying; receiving binding affinity data on each paired combination of one variant of the target peptide sequence and one variant of the targeting peptide sequence; identifying one or more synergistic pairs, wherein each synergistic pair includes one variant of the target peptide sequence and one variant of the targeting peptide sequence with binding affinity above a threshold; performing multiple sequence alignment with the one or more homolog pairs and the one or more synergistic pairs; and applying a structure prediction model to the multiple sequence alignment to predict a structure of a protein complex formed by the target peptide sequence and the targeting peptide sequence.

### BRIEF DESCRIPTION OF THE DRAWINGS

FIG. 1 is a block diagram illustrating the networking environment of an analytics system, according to one or more embodiments.

FIG. 2 is a block diagram illustrating the architecture of the analytics system of FIG. 1, according to one or more embodiments.

FIG. 3A is a flowchart illustrating generation of synthetic pairs for multiple sequence alignment (MSA), according to one or more embodiments.

FIG. 3B is a flowchart illustrating multiple sequence alignment (MSA) and structure prediction with synthetic augmentation, according to one or more embodiments.

FIG. 4 illustrates graph results of synthetic augmentation of MSA compared to a non-augmented approach, according to one or more example implementations.

FIG. 5 illustrates additional graph results of synthetic augmentation of MSA compared to other non-augmented approaches, according to one or more example implementations.

FIG. 6 illustrates predicted aligned error (PAE) of synthetic augmentation of MSA compared to a non-augmented approach, according to one or more example implementations.

FIG. 7 illustrates a matrix of color-coded affinities for all possible pairs of protein A variants and protein B variants generated by single-site mutagenesis (SSM).

FIG. 8 shows synergistic effects at the residue level by zooming in on a 19 by 19 matrix that represents variant pairs of protein A and protein B with modifications at two specific residues (Residue X and Residue Y).

FIG. 9 illustrates a matrix of color-coded affinities for all possible pairs of VHH72 variants and COV-1 RBD variants. On the left, a heatmap illustrates the number of synergistic mutations observed at each residue pair. The top three residue pairs by multiplicity are listed in the accompanying table on the right.

FIG. 10 provides DockQ scores of structural predictions of VHH72, CR3022 and Fab8 obtained with Chai-1 both with and without constraints with the top three residue pairs in FIG. 9.

FIG. 11 provides DockQ scores of structural predictions of VHH72, CR3022 and Fab8 obtained with AlphaFold with injection of the paired MSA features.

### DETAILED DESCRIPTION

### Overview

The present disclosure provides a method of predicting a structure of an interface between a target peptide and a targeting peptide. The method leverages synthetic pairs of variants of a target peptide and variants of a targeting peptide and their binding affinities measured by a high-throughput analysis. Synergistic pairs among the synthetic pairs are selected and multiple sequence alignment (MSA) of the selected pairs is performed to predict a structure of the protein complex formed with the target peptide and the targeting peptide or an interface between the target peptide and the targeting peptide. Structure prediction using MSA of the synergistic pairs provides for improved results, thereby paving the path for downstream analyses, e.g., small molecule design for molecular glues or antibody design.

In consequence, improved MSA and structure prediction of protein complexes can greatly enhance the efficiency of computational resources in various biomedical applications, including small molecule and biologic such as protein, peptide, RNA or DNA designs (e.g., antibody design). For the design of small molecules or molecular glues, accurate protein complex structure prediction enables the precise modeling of the target protein's structure and its interaction sites. This can inform the design of small molecules that can effectively bind and mediate protein-protein interactions, thereby acting as “molecular glues.” An accurate structure prediction reduces the need for extensive trial-and-error and high-throughput screening processes, saving considerable wet lab resources or computational resources.

In context of antibody design for enhanced targeting of antigens, improved MSA or structure prediction can facilitate the identification of critical antibody-antigen interaction sites and guide the design of antibodies with improved specificity and affinity. By enabling a more targeted approach to the in silico engineering of antibodies, these resources significantly increase computational efficiency, reducing the number and breadth of simulations required to identify promising antibody candidates. Ultimately, such advances assist in accelerating vaccine and therapeutic development pipelines while conserving valuable wet lab resources or computational resources.

### Definitions

The terms, “targeting protein” and “targeting peptide”, are used interchangeably herein to refer to a protein or a peptide that binds to other macromolecules. Example targeting proteins or targeting peptides include, but are not limited to: enzymes, ligase, antibodies, antigens, receptors, ligands, etc.

The terms, “target protein” or “target peptide”, are used interchangeably herein to refer to a protein or a peptide that can bind to a targeting protein or a targeting peptide.

The term, “variant”, refers to a peptide or protein that includes one or more modifications (e.g., a deletion, insertion, substitution, chemical modification, or a combination thereof) from a base peptide or base protein (e.g., a target peptide/protein or a targeting peptide/protein). A variant can be a homolog of the base peptide or base protein. A variant can be a naturally existing peptide or protein or an artificial or synthetic peptide or protein. In some cases, a variant can be a natural homolog. In some cases, a variant is an artificially generated pseudo-homolog (e.g., an AI-generated pseudo-homolog) of a based protein or peptide. In some embodiments, a variant has at least a minimum sequence identity (e.g., 40%, 50%, 60%, 70% or higher) to a base peptide or based protein.

The term “synthetic pairs of variants” used herein refers to a plurality of pairs of variants, where one or more variants within the plurality of pairs are a non-naturally occurring protein or peptide.

A “binding motif” refers to a portion or all of a protein or peptide that is involved in binding with the other protein or peptide.

A “synergistic pair” refers to a pair of peptides (or proteins), whose affinity cannot be adequately explained by the affinities of the individual peptides (or individual proteins) separately measured against their base peptide (or protein).

A “synergistic mutation pair” refers to a pair of modifications across an interface of two peptides (or proteins) that exhibit non-additive impact on binding affinities between the two peptides (or proteins) compared to the impact of the individual modifications themselves.

The term, “multiple sequence alignment”, as used herein refers to a method or process by which two or more biological sequences (e.g., DNA, RNA, or protein sequences) are arranged or aligned to identify regions of similarity. Such similarities may be indicative of functional, structural, or evolutionary relationships among the sequences. The alignment may be represented in a matrix format where residues or nucleotides that are evolutionarily conserved are aligned in columns, and gaps may be introduced to optimize the alignment.

The term, “mediating ligand”, as used herein refers to any molecule, macromolecule, or molecular entity that is capable of binding, associating, or interacting with both a target peptide and a targeting peptide. The mediating ligand can be but not limited to small organic or inorganic molecules, peptides, proteins, nucleic acids such as DNA or RNA (including aptamers), carbohydrates, lipids, synthetic polymers, or combinations or derivatives thereof.

### System Environment

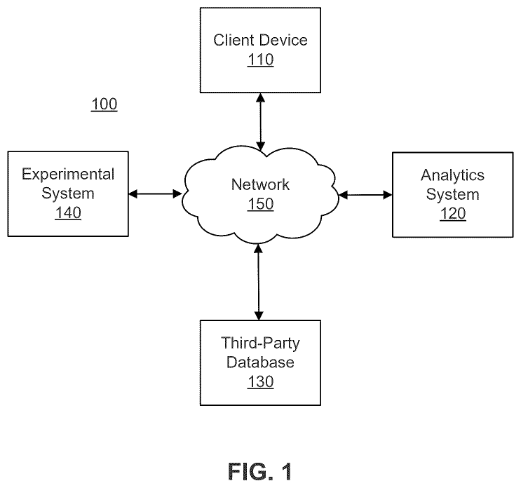

FIG. 1 illustrates an example system environment for an analytics system 130, in accordance with one or more embodiments. The system environment illustrated in FIG. 1 includes a client device 110, an analytics system 120, a third-party database 130, an experimental system 140, and a network 150. Alternative embodiments may include more, fewer, or different components from those illustrated in FIG. 1, and the functionality of each component may be divided between the components differently from the description below. Additionally, each component may perform their respective functionalities in response to a request from a human, or automatically without human intervention.

A client device 110 is a computing device operated by a user performing one or more computational analyses with the analytics system 120. The client device 110 is configured to receive inputs and to display results of analyses by the analytics system. Accordingly, the client device 110 is a computing device that interacts with other components in the system environment 100 via the network 140. In one or more embodiments, a user may provide to the client device 110 an input including a target peptide or a targeting peptide being analyzed. The client device 110 may relay the input to the analytics system 120 via the network 140 to explore interactions between the one or more peptides. In one or more embodiments, the client device 110 may provide the target peptide and the targeting peptide, e.g., as a query, to evaluate multiple sequence alignment or structure prediction. The analytics system 120 may relay the results of the computational analyses to the client device 110 for display to the user. The user may provide additional inputs to the client device 110 to perform additional computation analyses.

The analytics system 120 performs one or more computational analyses. The analytics system 120 is configured to receive inputs from the client device 110. In one or more embodiments, the analytics system 120 performs synthetic augmentation for multiple sequence alignment (MSA) and protein-protein structure prediction. In one or more embodiments, the analytics system 120 queries the third-party database 130 for homolog pairs to a target peptide and targeting peptide pair. The analytics system 120 further generates variants for the target peptide and the targeting peptide for wet lab experimentation. In one or more embodiments, the variants may include first order, second order, third order, or higher order mutations to the peptide sequences. The analytics system 120 provides the variants to the experimental system 140 for experimental assessment of binding affinity. With the experimental binding affinity, the analytics system 120 may identify synergistic pairs of mutated target peptides and targeting peptides with high binding affinity (e.g., above a threshold binding affinity). The analytics system 120 may incorporate both the homolog pairs (e.g., identified from the third-party database 130) and the synthetic pairs (e.g., the synergistic pairs of mutated target peptides and targeting peptides) to yield increased depth in multiple sequence alignment. The analytics system 120 may also perform protein-protein structure binding prediction. Through MSA and/or structure prediction, the analytic system 120 can identify residues on the target peptide and/or on the targeting peptide that contribute to the protein-protein interaction. In one or more embodiments, the analytics system 120 may leverage the synthetic augmentation of MSA for small molecule design as molecular glues. In other embodiments, the analytics system 120 may leverage the synthetic augmentation of MSA for antibody design in targeting particular antigens of interest.

The third-party database 130 is an online database that stores data, e.g., that may be retrieved and used by the analytics system 120. In one or more embodiments, the third-party database 130 stores data on one or more proteins. For example, the third-party database 130 may include data on experimental binding affinity between one or more peptides and a targeting protein. The third-party database 130 may further include other experimental data characterizing the one or more proteins. For example, other experimental data may include crystallography information, thermodynamic characteristics, chemical properties, other functional properties, protein folding structure, etc. Other example data may include homolog pairs of protein-protein pairs identified and characterized from biological organisms (e.g., including from other species).

The experimental system 140 is a platform for performing one or more experiments. In one or more embodiments, the experimental system 140 is used to manufacture and experiment with proteins. In some embodiments, the experimental system 140 may be a human-operated laboratory environment. In other embodiments, the experimental system 140 may be an automated platform with one or more devices for conducting one or more experiments on proteins. For example, the experimental system 140 may manufacture proteins and performing screening assays to assess binding affinity. The experimental system 140 may operate autonomously (i.e., devices and/or robots that are computer driven perform the experiments), manually (i.e., human operator controls the devices and/or robots), or semi-autonomously (i.e., human operator works in conjunction with automated devices and/or robots).

In one or more embodiments, the experimental system 140 may include one or more devices for manufacturing proteins for the experiments. Such devices may include a DNA synthesis device for manufacturing DNA molecules for coding a target protein, a protein synthesis device for protein expression with the synthetically generated DNA molecules, and one or more measurement devices for measuring characteristics of the manufacture proteins. The DNA synthesis device may implement chemical synthesis to create the DNA molecules. Chemical synthesis is a solid-phase phosphoramidite chemical process. In chemical synthesis, the desired DNA sequence is built step-by-step by adding one nucleotide at a time. The process occurs on a solid support, usually a controlled pore glass bead, where the first nucleotide is attached. The synthesis proceeds using a series of reactions to add each subsequence nucleotide successively. This method can produce DNA molecules, e.g., up to 200 base pairs long. These synthesized DNA molecules can be assembled into larger constructs. The one or more devices may alternatively manufacture other molecules. For example, the devices may produce a mediating ligand for use in experiments for assaying binding affinity, e.g., between a targeting peptide and a target peptide. The protein synthesis device may be configured to transfect a cell line with the synthetically generated DNA molecules. Example cell lines include bacteria, yeast, and mammalian cells. The choice of host cell system depends on factors such as scalability, cost, and compatibility with the protein's structure and function. The transfected cell lines are maintained to produce the protein through the cell's natural functions. Following protein expression, the experimental system 140 may perform protein extraction and purification to yield a high-quality and functional protein product. Common purification methods include affinity chromatography, ion exchange chromatography, size exclusion chromatography, and precipitation. The end result is the extracted and purified target protein.

In one or more embodiments, the experimental system 140 comprises one or more devices for measuring characteristics of the manufactured proteins. These measurements devices may perform one or more wet lab analyses on the protein manufactured. Wet lab analyses aim to characterize or to validate the manufactured protein. For example, the experimental system 140 may sequence the manufactured protein to determine whether the manufactured protein matches to the intended target protein. In other examples, the experimental system 140 may characterize the structure of the manufactured protein, e.g., through x-ray crystallography. The experimental system 140 may further run experiments with the manufactured protein while measuring characteristics, e.g., denaturing the manufacture protein to determine refolding structure, etc.

In one or more embodiments, the experimental system 140 includes one or more devices for measuring binding affinity between one or more peptides and a targeting protein. The experimental system 140 may grow a first culture expressing the one or more peptides and a second culture expressing the targeting protein. For example, the cultures may be yeast cultures expressing the peptides or the targeting protein on a surface of yeast cells. The experimental system 140 combines the first culture and the second culture to permit mating events between yeast cells expressing the one or more peptides in the first culture and yeast cells expressing the targeting protein in the second culture. The experimental system 140 may determine a binding affinity based on counts of mating events for each combination of one peptide and the targeting protein.

The client device 110, the analytics system 120, the third-party database 130, and the experimental system 140 can communicate with each other via the network 150. The network 150 is a collection of computing devices that communicate via wired or wireless connections. The network 150 may include one or more local area networks (LANs) or one or more wide area networks (WANs). The network 150, as referred to herein, is an inclusive term that may refer to any or all of standard layers used to describe a physical or virtual network, such as the physical layer, the data link layer, the network layer, the transport layer, the session layer, the presentation layer, and the application layer. The network 150 may include physical media for communicating data from one computing device to another computing device, such as multiprotocol label switching (MPLS) lines, fiber optic cables, cellular connections (e.g., 3G, 4G, or 5G spectra), or satellites. The network 150 also may use networking protocols, such as TCP/IP, HTTP, SSH, SMS, or FTP, to transmit data between computing devices. In some embodiments, the network 150 may include Bluetooth or near-field communication (NFC) technologies or protocols for local communications between computing devices. The network 150 may transmit encrypted or unencrypted data.

### Analytics System Architecture

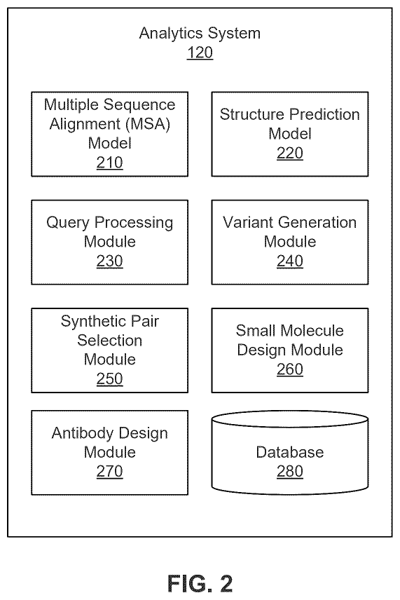

FIG. 2 is a block diagram illustrating the architecture of the analytics system 120 of FIG. 1, according to one or more embodiments. The analytics system 120 includes a multiple sequence alignment (MSA) model 210, a structure prediction model 220, a query processing module 230, a variant generation module 240, a synthetic pair selection module 250, a small molecule design module 260, an antibody design module 270, and a database 280. The analytics system 120 may include additional, fewer, or different components than those listed herein FIG. 2. In other embodiments, functions of each component can be disparately distributed throughout the components of the analytics system 120. In other embodiments, functionality described herein may be performed by another entity, system, or third-party, e.g., in conjunction with the analytics system 120.

The multiple sequence alignment (MSA) model 210 aligns multiple peptide sequences to identify regions of similarity. The regions of similarity may be the result of shared ancestries between the peptide sequences or may be common functional, structural, or evolutionary relationships. In one or more embodiments, the MSA model 210 performs sequence weighting to account for redundancy of strings of sequences across the multiple peptide sequences. The MSA model 210 may further assign position-specific gap penalties to account for naturally occurring gaps. The MSA model 210 outputs a matrix that highlights the aligned regions of similarity across the peptide sequences.

In one or more embodiments, the MSA model 210 is a machine-learning model that is trained on annotated sequence sets. In such embodiments, the analytics system 120 may train the MSA model 210 by inputting the annotated sequence sets, predicting alignment, scoring the prediction against the annotations, and adjusting parameters of the MSA model 210 to optimize the scoring.

The MSA model 210 is configured to input pairs of peptide sequences to output an alignment between the pairs of peptide sequences. In one or more embodiments, the analytics system inputs variants of a targeting peptide and variants of a target peptide in conjunction with the targeting peptide and the target peptide into the MSA model 210 to perform multiple sequence alignment. The variants may include homolog pairs. The homolog pairs may be obtained from third-party databases, or identified through extraction and sequencing from living organisms. In some embodiments, the homolog pairs are synthetically or artificially generated by a generative artificial intelligence model. In performing the multiple sequence alignment, the MSA model 210 may perform separate alignments, e.g., one alignment for the targeting peptide and its variants, and another alignment for the target peptide and its variants.

In one or more embodiments, the generative artificial intelligence model may be a transformer-based model, e.g., a protein language model. To train the generative artificial intelligence model, the analytics system may obtain a large corpus of protein or peptide sequences. The analytics system masks certain tokens in the sequences, then prompts the model to infill the masked tokens. The analytics system scores the predictions against the unmasked sequences. The analytics system may further fine tune the model for various inference tasks, such as the generative task of synthetically generating homolog pairs for a targeting peptide and a target peptide.

The structure prediction model 220 inputs aligned peptide sequences (e.g., the output of the MSA model 210) to predict structural configuration of the peptide sequences. In one or more embodiments, the structure prediction model 220 may input pair(s) of peptide sequences of a targeting peptide and a target peptide (e.g., which may be in matrix form aligned by the MSA model 210). The structure prediction 220 determines pairwise distances of residues in the peptide sequence(s) or angles between residues. With the pairwise distances or the angles, the structure prediction model 220 may output a three-dimensional spatial model of the peptide sequence(s) (e.g., the protein complex formed by the targeting peptide and the target peptide). Specifically in the context of a protein complex, the structure prediction model 220 may output a joint distance map for residues and/or heavy atoms on both peptide sequences. In one or more embodiments, the structure prediction model 220 may output spatial coordinates for the residues and/or the heavy atoms on both peptide sequences. In some embodiments, the structure prediction model 220 further inputs one or more mediating ligands (e.g., the chemical formula of the mediating ligand). The structure prediction model 220 may predict the structure of the entire complex, inclusive of the three or more molecules.

In one or more embodiments, the structure prediction model 220 is a template-based model leveraging known protein or peptide structures, e.g., stored locally or in third-party databases. In such embodiments, the structure prediction model 220 may leverage the multiple sequence alignment to identify homologs or similar protein folding structures to the targeting peptide and the target peptide. In one or more embodiments, the structure prediction model 220 may use the known structures of any identified homologs as a baseline template to determine the structure for the targeting peptide and the target peptide. In one or more embodiments, the structure prediction model 220 may score similarity of the targeting peptide sequence and the target peptide sequence to known structural folds and their sequences. Leveraging this information, the structure prediction model 220 can craft the structure prediction by piecing similar folds together.

In one or more embodiments, the structure prediction model 220 leverages physics and conformational sampling to determine the structure. In such embodiments, the structure prediction model 220 may leverage one or more energy functions and a sampling algorithm to traverse the high-dimensional space. As the structure prediction model 220 traverses the high-dimensional space, the structure prediction model 220 identifies one or more conformations resulting in minima energy states, suggesting the stabilized structure of the protein complex.

In one or more embodiments, the structure prediction model 220 is a machine-learning model, e.g., a deep-learning model. The structure prediction model 220 may be trained with training data to predict the protein structure. In some embodiments, the structure prediction model 220 is trained in a supervisory fashion. Training data involving amino acid sequences and their known structure (e.g., as determined through x-ray crystallography or other imaging techniques) are fed into the structure prediction model 220 for deep-learning of how proteins or peptides fold. Outputs of the structure prediction model 220 may be scored against the known structure (i.e., as ground truth). Parameters or weights of the structure prediction model 220 are tuned to steer the outputs towards the ground truth, e.g., via a backpropagation algorithm. In some embodiments, the structure prediction model 220 performs unsupervised or self-supervised learning. In such embodiments, the structure prediction model 220 is trained with data that is unlabeled, e.g., multiple sequence alignment data provides insights into residues that have co-evolved over time, suggesting proximity to the binding interface. In some embodiments, the structure prediction model 220 may be trained in a hybrid approach, wherein some portion of the training data is labeled with ground truth and another portion of the training data is unlabeled.

In one or more embodiments, the structure prediction model 220 can identify amino acid residues in the targeting peptide and/or the target peptide that are part of the binding interface. In one or more embodiments, the structure prediction model 220 may identify amino acid residues across the interface within some threshold distance, e.g., 4 Angstroms, 5 Angstroms, 6 Angstroms, 7 Angstroms, 8 Angstroms, 9 Angstroms, or 10 Angstroms. In some embodiments, the structure prediction model 220 may be configured to output an indication of which residues are part of the binding interface. For example, the structure prediction model 220 may be trained to output a likelihood per residue predicting whether the residue is component to the binding interface. The structure prediction model 220 can leverage a threshold likelihood to predict which residues are part of the binding interface. The structure prediction model 220 may tag the residues predicted to be component to the binding interface. In one or more embodiments, the analytics system 200 may generate a graphical representation of the structure prediction. The graphical representation may further visually distinguish the residues predicted to be component to the binding interface. This functionality empowers a researcher to easily identify these residues.

In one or more embodiments, the structure prediction model 220 is configured to output a confidence score associated with the structure prediction. The structure prediction model 220 may further output a confidence associated with the spatial positioning of each unit in the protein complex (e.g., the amino acid residues, or the heavy atoms in the peptide sequences). In one or more embodiments, the confidence may be calculated based on a consensus of the unit's positioning based on neighboring units in the complex. In one or more embodiments, the confidence may be calculated based on an energy function. For example, one residue which contributes an above-average energy to the aggregate energy of the complex may yield a lower confidence compare to residues contributing at or below the average energy.

The query processing module 230 receives a query including a targeting peptide and a target peptide, e.g., to perform MSA or structure prediction. The query processing module 230 obtains homolog pairs of the targeting peptide and the target peptide, e.g., in the database 280 or the third-party database 130.

The variant generation module 240 generates variants of the targeting peptide and the target peptide, e.g., provided in the query from the client device 110. In one or more embodiments, a variant may include a first order, second order, third order, or other higher order mutation. The order of mutation refers to the number of mutations of residues on a peptide sequence. For example, a second order mutation includes at least two mutated residues on the peptide sequence. The variant generation module 240 may enumerate all possible variants up to a certain order of mutation. In other embodiments the variant generation module 240 may generate a random subset of possible variants. For example, the variant generation module 240 may determine one thousand random first order mutation variants, one thousand random second order mutation variants, etc. The variant generation module 240 provides the variants of the targeting peptide and the target peptide to the experimental system 140 for wet lab experimentation to assess binding affinity between the variants. In one or more embodiments, the variant generation module 240 may further generate variants of the targeting peptide and the target peptide of homolog pairs of the query peptides. The variant generation module 240 may provide the variants to the experimental system 140 to assess binding affinity between each paired combination of a variant targeting peptide and a variant target peptide. In one or more embodiments, the variant generation module 240 may generate over 100 variants, 200 variants, 300 variants, 400 variants, 500 variants, 600 variants, 700 variants, 800 variants, 900 variants, 1,000 variants, 2,000 variants, 3,000 variants, 4,000 variants, or 5,000 variants per peptide (i.e., for the targeting peptide and/or for the target peptide).

In one or more embodiments, variant generation module 240 may perform an iterative process in generating variant for the targeting peptide or the target peptide. The variant generation module 240 may further iterate on synergistic pairs of a variant targeting peptide and a variant target peptide. For example, the variant generation module 240 may generate an initial batch of first order variants, i.e., variant targeting peptides and variant target peptides. Upon identifying a synergistic pair from the first batch, the variant generation module 240 may generate a subsequent batch of second order variants based on the synergist pair, i.e., mutating other residues of the synergistic pair. The variant generation module 240 may also provide the subsequent batch of second order variants to the experimental system 140 for assessing binding affinity. In some embodiments, the variant generation module 240 may leverage an optimization algorithm to traverse the high-dimensional search space to iterate on the variant generation process. In one or more embodiments, the variant generation module 240 leverages a fitness function for defining the fitness of the variant targeting peptide and/or the variant target peptide. The fitness function could output likelihood of the two being synergistic. In other embodiments, the fitness function could output binding affinity of the two. The module may implement Bayesian optimization by building probabilistic models of the fitness landscape to guide sampling of the landscape. The probabilistic models may inform residues to iterate the mutation process. In one or more embodiments, the module may implement reinforcement learning, leveraging multiple agents to simultaneously sample the fitness landscape. Agents that identify promising variants, i.e., that trend towards optima of the fitness landscape, are rewarded. In other embodiments, the module may leverage generative machine-learning models to guide the sampling of variants. The machine-learning models may be deep-learning neural networks, transformer-based models, etc. In some embodiments, other optimization techniques can be applied, e.g., simulated annealing, Monte Carlo sampling, etc. The variant generation module 240 may continue the iterative sampling process until one or more stopping conditions are met. One stopping condition may be identifying a sufficient number of synergistic pairs. For example, upon identifying 2, 3, 4, 5, 6, 7, 8, 9, or 10 synergistic pairs, the variant generation module 240 stops the iterative process. Another stopping condition may be identifying synergistic pairs achieving target parameters, e.g., achieving a non-additive effect above a threshold value, achieving a binding affinity above a threshold, etc. Yet another stopping condition may be exhausting some budget on the sampling, e.g., iterating on the sampling process 3, 4, 5, 6, 7, 8, 9, or 10 instances.

The synthetic pair selection module 250 identifies synergistic pairs of a variant targeting peptide and a variant target peptide based on binding affinity data. The experimental system may provide a matrix of binding affinity between the variants of the targeting peptide and the variants of the target peptide, with each paired combination characterized by an experimental binding affinity. In one or more embodiments, synergistic pairs are pairs where the experimental binding affinity of the targeting peptide variant vs. the target peptide variant is non-additive, e.g., higher than expected, based on the experimental binding affinity of the targeting peptide variant vs. the target peptide, and the targeting peptide variant vs. the target peptide. In a further example, the non-additive effect on binding affinity may include compensatory binding. In such a scenario, the variant targeting peptide ablates binding against the wildtype target peptide and/or the variant target peptide ablates binding against the wildtype targeting peptide. The compensatory effect occurs when the variant targeting peptide and the variant target peptide rescues the binding that would otherwise be ablated by either or both variants when screened individually. In one or more embodiments, to measure synergy between a variant targeting peptide and a target peptide, the synthetic pair selection module 250 determines a score indicating the combinative effect on binding affinity the variant targeting peptide and the variant target peptide have when compared against their individual effect on binding affinity against their wildtype counterpart. In comparing the combinative effect, the synthetic pair selection module 250 may evaluate the matrix of binding affinity between the variants of the targeting peptide against the variants of the target peptide. The matrix be sized greater than 100-by-100, yielding over 10,000 data points on binding affinity. To measure specificity, the module scores each interaction based on desired effect and/or undesired effect. The synthetic pair selection module 250 may determine a specificity ratio measured by on-target interactions to off-target interactions. The synthetic pair selection module 250 may determine a discrimination score indicating the capability of each peptide to differentiates its cognate target from the strongest off-target. The synthetic pair selection module 250 may determine an area under the curve (AUC) from receiver operating characteristic analysis, describing a peptide's ability to distinguish cognate from non-cognate interactions. The synthetic pair selection module 250 use any of these metrics or combinations thereof in scoring synergy between a variant targeting peptide and a variant target peptide.

In other embodiments, to measure synergy, the synthetic pair selection module 250 may use a threshold on binding affinity. A high threshold would yield more confident binding pairs, but potentially lead to low numbers of synergistic pairs. A low threshold would yield less confident binding pairs, but provide larger quantity of such synergistic pairs. The synthetic pair selection module 250 may tune the threshold to balance objectives.

The small molecule design module 260 designs one or more small molecules to act as molecular glue between a target peptide and a targeting peptide. The small molecule design module 260 may receive structure information (e.g., output by the structure prediction model 220) of the protein complex. The small molecule design module 260 may determine the hole between the target peptide and the targeting peptide based on the structure information. The small molecule may be designed to mimic a structure of the hole. In one or more embodiments, the small molecule design is provided to the experimental system 140 to manufacture the small molecule to perform experimental validation of the gluing efficacy of the small molecule between the peptide of interest and the targeting protein. The small molecule may be smaller than 2,000 Daltons, 1,750 Daltons, 1,500 Daltons, 1,250 Daltons, 1,000 Daltons, 750 Daltons, 500 Daltons, or 250 Daltons.

In one or more embodiments, the small molecule design module 260 may iterate on the small molecule design. In iterating on the design, the small molecule design module 260 may experimentally validate the small molecule design. Based on the experimental results, the small molecule design module 260 may continue toggling design of the small molecule (e.g., modifying units, adding units, deleting units, rearranging units, or some combination thereof). The small molecule design module 260 then reevaluates the iterated design with wet laboratory experiments.

In one or more embodiments, the small molecule design module 260 may be applied to structure-activity relationship (SAR) studies of compound-mediated ternary complexes. In such scenarios, the small molecule is evaluated for its effect on forming ternary complexes with two proteins, e.g., a targeting peptide and a target peptide. Improved structure prediction of the ternary complexes without experimental structure prediction (which is often a difficult undertaking given the complexity of forming stable crystalline structures of the ternary complexes, e.g., for x-ray crystallography) empowers the molecule glue design process. This significantly streamlines the molecule glue design process, minimizing dependence on experimentally-determined structure of the protein complexes.

The antibody design module 270 designs one or more antibodies to target antigens of interest. The antibody design module 270 may leverage insights into the predicted structure and associated confidence (e.g., output by the structure prediction model 220) to design antibodies to improve targeting capabilities. The small molecule may be of a size on the order of 100 kiloDaltons, 200 kiloDaltons, 300 kiloDaltons, 400 kiloDaltons, 500 kiloDaltons, 600 kiloDaltons, 700 kiloDaltons, 800 kiloDaltons, 900 kiloDaltons, or 1,000 kiloDaltons.

The database 280 stores data used by the analytics system 120 and its various components. For example, the database 280 may store the parameters of the MSA model 210, the structure prediction model 220. The database 270 may also store all the generated variants, synthetic pairs, experimental binding affinity data received by the experimental system 140, or some combination thereof. The database 270 may also store other protein information on various peptides, e.g., as retrieved by the third-party database 130 or determined by components of the analytics system 120.

In one or more embodiments, the antibody design module 270 may iterate on the antibody design. In iterating on the design, the antibody design module 270 may experimentally validate the antibody design, e.g., assaying binding affinity against a target antigen. Based on the experimental results, the antibody design module 270 may continue toggling design of the antibody (e.g., modifying residues, adding residues, deleting residues, rearranging residues, or some combination thereof). The antibody design module 270 then reevaluates the iterated design with wet laboratory experiments.

In one or more embodiments, the antibody design module 270 designs the antibody informed by residues predicted to be part of the binding interface. In such embodiments, the antibody design module 270 can selectively toggle residues. For example, in one or more example implementations, the antibody design module 270 toggles or modifies residues not component to the binding interface so as to preserve the current binding affinity of the antibody to the antigen. This is advantageous in trying to tune other characteristics of the antibody design, e.g., to improve solubility, to improve manufacturability, etc. In other example implementations, the antibody design module 270 toggles or modifies residues component to the binding interface, so as to tune the binding affinity of the antibody to the antigen.

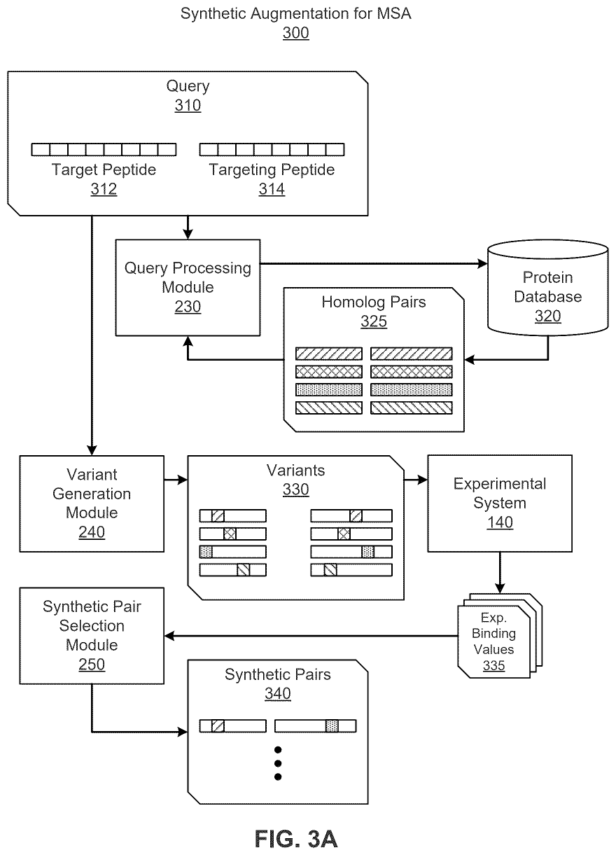

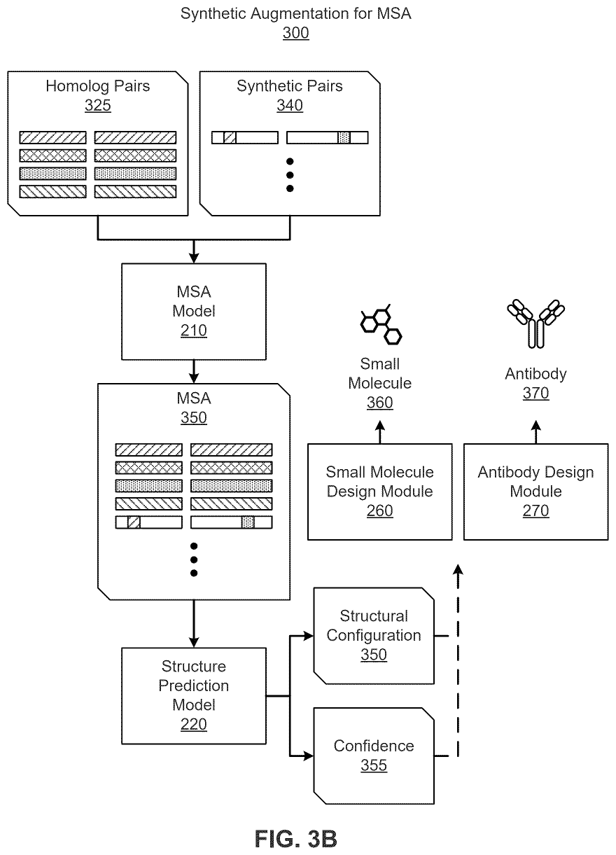

FIGS. 3A & 3B illustrate synthetic augmentation of MSA or structure prediction of a query including a target peptide and a targeting peptide. The steps described may be accomplished by the client device 110, the analytics system 120 (and its components), the third-party database 130, the experimental system 140.

The analytics system 120 receives a query 310 including target peptide 312 and a targeting peptide 314 (e.g., from a client device). The target peptide 312 and the targeting peptide 314 may be queried to design small molecule(s) to act as molecular glues in catalyzing the binding of the target peptide 312 and the targeting peptide 314.

The query processing module 230 queries the protein database 320 for homolog pairs 325 of the queries target peptide 312 and the targeting peptide 314. These homolog pairs may be identified by other researchers as being homologs.

The variant generation module 240 generates variants 330 of the target peptide 312 and the targeting peptide 314. For example, the variants may include first order, second order, third order, or other higher order mutations. The variants generation module 240 provides the variants to the experimental system 140 to perform binding affinity assays between paired combinations of a variant target peptide and a variant targeting peptide. The experimental system 140 may provide the experimental binding values 335 for the paired combinations to the synthetic pair selection module 240. The synthetic pair selection module 250 selects the synthetic pairs with high binding affinity, i.e., having a binding affinity above a threshold. These synthetic pairs may not have been previously documented (or otherwise known) as naturally occurring in biological organism. In other words, these synthetic pairs may be non-naturally-occurring pairs. This is further frustrated by limitations of technologies to measure binding activity with diversity on both sides of a protein-protein interaction.

The analytics system 120 feeds the homolog pairs 325 and the synthetic pairs 340 (e.g., with high binding affinity) to the MSA model 210. The MSA model 210 outputs an alignment of the multiple sequences (i.e., MSA 350), i.e., of the peptide sequences in the homolog pairs 325 and the peptide sequences in the synthetic pairs 340. The analytics system 120 may then input the MSA 350 into the structure prediction model 220 to output a predicted structural configuration 350 for the protein complex formed by the target peptide and the targeting peptide and, optionally, a confidence value 355 associated with the structural configuration 350.

The analytics system 120 may leverage the structural configuration 350 or the confidence 355 to inform small molecule design or antibody design. For example, the small molecule design module 260 may leverage the structural configuration 350 or the confidence 355 to identify putative holes between the target peptide and the targeting peptide to inform small molecule 360 design. As another example, the antibody design module 270 may leverage the structural configuration 350 or the confidence 355 to identify residue sequences that optimally target antigens of interest in antibody 370 design.

### Method of Predicting a Structure of an Interface Between a Target Peptide and a Targeting Peptide

In one aspect, the present disclosure provides a method of generating a predicted structure of an interface between a target peptide and a targeting peptide.

The method may be performed by a computer system, e.g., the analytics system 120. In one or more embodiments, one or more steps may be performed by another entity, e.g., a human, another computer system, an experimental system, a third-party system, etc. In other embodiments, one or more steps may be optional to the method.

The method comprises obtaining sequences of (i) a first library of variants of the target peptide and (ii) a second library of variants of the targeting peptide. The first library of variants of the target peptide may have one or more homologs of the target peptide and the second library of variants of the targeting peptide may have one or more homologs of the targeting peptide. The one or more homologs of the target peptide and/or the one or more homologs of the targeting peptide may be generated by a generative machine-learning model. The plurality of test pairs may include one or more pairs of a homolog of the target peptide and a homolog of the targeting peptide.

The method comprises generating a plurality of test pairs. Each test pair may include one target variant selected from the variants of the target peptides and one targeting variant selected from the variants of the targeting peptide. The method comprises obtaining binding affinity of the test pairs. In some embodiments, the method further comprises sending the test pairs for experimentation to assay binding affinity. In some embodiments, the binding affinity data is obtained for database.

In some embodiments, the method comprises obtaining binding affinity data for each of the plurality of test pairs. In some embodiments, the binding affinity data is obtained by a high-throughput analysis of binding between the test pairs. The high-throughput analysis may entail: expressing variants of the target peptide in the first library and variants of the targeting peptide in the second library on surfaces of two separate haploid strains of yeasts; and measuring rates at which yeasts of two separate haploid strains fuse into diploids, thereby obtaining the binding affinity data. In some embodiments, the high-throughput analysis is performed in the presence of a mediating ligand. In some embodiments, the binding affinity data indicate binding affinity among the variants of the target peptide, the variants of the targeting peptide, and the mediating ligand.

In some embodiments, the method comprises selecting one or more pairs out of the test pairs as one or more synergistic pairs based on the binding affinity data. In some embodiments, the one or more synergistic pairs are selected from test pairs for being a synergistic mutation pair, i.e., having non-additive impact on binding affinities between the two peptides compared to the impact of the individual modifications themselves. In one or more embodiments, the one or more synergistic pairs are selected from test pairs for having a binding affinity above a threshold, e.g., which may be based on the binding affinity between the non-mutated targeting peptide and the non-mutated target peptide. In other embodiments, the one or more synergistic pairs are selected from test pairs for having a binding affinity below a threshold, e.g., which may be based on the binding affinity between the non-mutated targeting peptide and the non-mutated target peptide.

In some embodiments, the method further comprises performing multiple sequence alignment with the sequences of target variants and targeting variants in the one or more synergistic pairs. Performing the multiple sequence alignment may entail: performing sequence alignment of sequences of the targeting variants in the one or more synergistic pairs; and performing sequence alignment of sequences of the target variants in the one or more synergistic pairs. Multiple sequence alignment may leverage a function that scores the alignment between sequences. A model finds an optimum of the function, e.g., that maximizes likelihood that the alignment

In some embodiments, the method comprises generating the predicted structure of the interface between the target peptide and the targeting peptide based on the multiple sequence alignment. The predicted structure may be a spatial model of the protein binding complex. The spatial model may include spatial coordinates for units of the protein complex (e.g., residues, molecules, heavy atoms, etc.). In some embodiments, the predicted structure of the interface is a structure of the interface in the presence of the mediating ligand between the target peptide and the targeting peptide. The predicted structure of the interface may also include a structure of the mediating ligand.

In one or more embodiments, the method comprises identifying amino acid residues in the targeting peptide and amino acid residues in the target peptide that contribute to interaction between the targeting peptide and the target peptide based on the multiple sequence alignment. In some embodiments, the method comprises generating the predicted structure of the interface based on the identified amino acid residues in the targeting peptide and the identified amino acid residues in the target peptide that contribute to interaction between the targeting peptide and the target peptide. Generation of the predicted structure may include using the identified amino acid residues in the targeting peptide and the identified amino acid residues in the target peptide as constraints to the spatial model.

In one or more embodiments, the method comprises applying a structure prediction model to generate the structure of the interface. The structure prediction model may be configured as a machine-learning model, configured to input the multiple sequence alignment to predict the structure of the interface. The structure prediction model may be trained on multiple sequence alignments of natural protein sequences. In some embodiments, the natural protein sequences comprise natural homologs of the targeting peptide and/or the target peptide. In some embodiments, the structure prediction model is configured to constrain structure prediction by establishing the amino acid residue residues in the targeting peptide and the amino acid residues in the target peptide as component to the binding interface of the targeting peptide and the target peptide.

In one or more embodiments, the system determines a confidence evaluation associated with the predicted structure of the interface. In some embodiments with the structure prediction model, the structure prediction model may be trained to further output the confidence evaluation in conjunction with the predicted structure. The system may determine the confidence evaluation further based on any identified synergistic pairs, residues identified as component to the binding interface, or some combination thereof. Leveraging this data grounds the model's confidence evaluation to binding affinity data between the variants of the targeting peptide and the variants of the target peptide. The confidence evaluation may be represented by a PAE score.

In one or more embodiments, the method may leverage the structure prediction to design a mediating ligand to facilitate binding between the targeting peptide and the target peptide. In some embodiments, the method comprises generating a structure of a mediating ligand or selecting a mediating ligand that can facilitate binding between the target peptide and the targeting peptide using the predicted structure of the interface between the target peptide and the targeting peptide. In some embodiments, the method comprises evaluating the mediating ligand. To do so, in some embodiments, the method comprises generating the mediating ligand. In some embodiments, the method comprises testing a binding affinity of the target peptide and the targeting peptide in the presence of the mediating ligand. In some embodiments, the method comprises iterating by modifying the mediating ligand or selecting an alternative ligand to improve binding to the target peptide and/or the targeting peptide.

In one or more embodiments, the method comprises designing the targeting peptide based on the structure prediction. In some embodiments, the method comprises identifying a first set of amino acid residues of the targeting peptide contribute to binding to the target peptide based on the structure of the interface and a second set of amino acid residues of the targeting peptide that do not contribute to binding to the target peptide sequence. In some embodiments, the method comprises generating an investigative variant of the targeting peptide by modifying one or more amino acid residues of the second set of amino acid residues of the targeting peptide sequence. In some embodiments, the method comprises evaluating the binding of the investigative variant by performing a binding affinity assay. In some embodiments, the method comprises receiving binding affinity data on the investigative variant of the targeting peptide and the target peptide. In some embodiments, the method comprises determining that the binding affinity data on the investigative variant of the targeting peptide sequence and the target peptide sequence is advantageous, e.g., achieves certain design parameters, is greater than a threshold, etc. In some embodiments, the method comprises assessing other characteristics of the investigative variant to optimize the design. In some embodiments, the method comprises producing the investigative variant of the targeting peptide, e.g., for formulating treatments to individuals for various diseases. In some embodiments, the targeting peptide is an antibody, and the target peptide is an antigen.

In one or more embodiments, the method comprises generating a digital representation of the structure of the binding interface between the target peptide and the targeting peptide. In some embodiments, the method comprises providing the graphical user interface to a client device, e.g., for display on the client device. In some embodiments, the method comprises generating the graphical user interface presenting the digital representation of the structure of the binding interface between the target peptide and the targeting peptide by: tagging, in the digital representation, the amino acid residues in the targeting peptide sequence and the amino acid residues in the target peptide sequence that contribute to binding of the targeting peptide and the target peptide.

### Experimental Analysis of Binding Affinity

In various embodiments, an experimental binding affinity value between two proteins (e.g., a variant of a target peptide and a variant of a targeting peptide) or between a targeting peptide and a target peptide is used. The binding affinity can be measured by various method known in the art. In some embodiments, an experimental approach for analyzing binding affinity of a library comprising a plurality of peptides is used. In some embodiments, a high-throughput analysis method is used. In some embodiments, the binding affinity is measured by protein-protein interaction screening approaches, e.g., phage display or yeast surface display. Some embodiments use binding affinity data obtained by the method described in U.S. Pat. No. 11,466,265, which is incorporated by reference.

In some embodiments, a high-throughput analysis is performed by a method comprising: expressing variants of the target peptide in the first library and variants of the targeting peptide in the second library on surfaces of two separate haploid strains of yeasts; and measuring rates at which yeasts of two separate haploid strains fuse into diploids, thereby obtaining the binding affinity data. In some embodiments, the binding affinity data is obtained by a method which comprises providing one or more targeting proteins expressed and displayed on the surface of a first plurality of recombinant haploid yeast cells; providing a plurality of target peptides expressed and displayed on the surface of a second plurality of recombinant haploid yeast cells, wherein the plurality of target peptides comprises a library of wild-type polypeptide substrate species and mutant polypeptide substrates species that have been modified at one or more amino acid residue positions by mutagenesis; combining the first plurality of recombinant haploid yeast cells and the second plurality of recombinant haploid yeast cells in a liquid medium to produce a culture; growing the culture for a time and under conditions such that one or more interactions between one or more of targeting proteins and one or more of the target peptides mediates one or more mating events between one or more of the first plurality of recombinant haploid yeast cells and one or more of the second plurality of recombinant haploid yeast cells to produce one or more diploid yeast cells; and determining, based on the number of mating events in the culture, the strength of the interactions between one or more of the targeting protein and one or more of the target peptides.

In further embodiments, the strength of the interaction (KD) between the targeting protein and the target peptide is stronger or weaker than the interaction between the targeting protein and the corresponding wild-type target peptide.

In some embodiments, the targeting protein is a ligase. In some embodiments, the targeting protein is a E3 ubiquitin ligase species. E3 ubiquitin ligases include MDM2, CRL4CRBN, SCFB-TrCP, UBE3A, and many other species that are well known in the art. E3 ubiquitin ligases recruit the E2 ubiquitin-conjugating enzyme that has been loaded with ubiquitin, recognize its target protein substrate, and catalyze the transfer of ubiquitin molecules from the E2 to the protein substrate for subsequence degradation by the proteasome complex.

In some embodiments, the targeting protein is a receptor, an antibody, or a modification thereof.

In some embodiments, the one or more target peptides comprise a known or predicted binding motif of the targeting protein. In some embodiments the one or more target peptides comprise a degron motif. In other embodiments, one or more of the target peptides have been modified at one or more amino acid residue positions by mutagenesis.

In other embodiments, the method further comprises computationally modeling the interface between the targeting protein and the target peptide. In some embodiments, the target peptide has been modified at one or more amino acid residue positions by mutagenesis in order to determine the structure of the interface between the targeting protein and the target peptide. In further embodiments, the method further comprises growing the culture in the presence of one or more small molecules, proteins, peptides, pharmaceutical compound, or other chemical entities.

In some embodiments the binding affinity is measured in the presence of a mediating ligand. In the case, the binding affinity can indicate a binding affinity between the two peptides in the presence of the mediating ligand.

In yet other embodiments, the method comprises identifying pairs of a target protein and target peptides wherein the strength of their interaction (KD) is stronger or weaker in the presence of one or more small molecules, proteins, peptides, pharmaceutical compound, or other chemical entities compared to their interaction in the absence of the one or more small molecules, proteins, peptides, pharmaceutical compound, or other chemical entities.

### Example 1

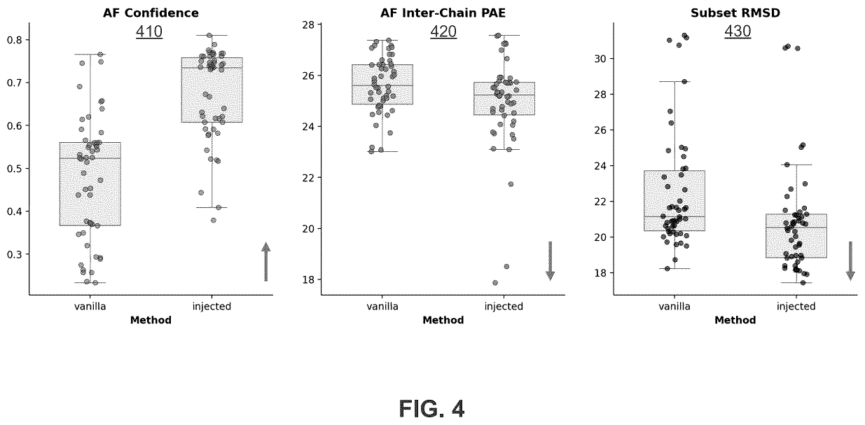

FIGS. 4 & 5 illustrate example graph results of synthetic augmentation of MSA or structure prediction.

FIG. 4 illustrates graph results of synthetic augmentation of MSA or structure prediction compared to a non-augmented approach, according to one or more example implementations. In these example implementations, the analytics system takes 50 queries of paired target peptide and targeting peptide. In a first approach, the analytics system performs MSA and structure prediction without synthetic augmentation. In this first approach, the analytics system identifies homolog pairs in protein databases. The analytics system performs MSA and structure prediction with the homolog pairs. In a second approach the analytics system performs synthetic augmentation of MSA and structure prediction (e.g., as described in FIGS. 3A & 3B). The analytics system identifies homolog pairs and screens for synthetic pairs of variants of the target peptide and variants of the targeting peptide with high binding affinity (e.g., as determined by a binding affinity assay performed by the experimental system 140). The analytics system performs the MSA and the structure prediction with the homolog pairs and the synthetic pairs.

The first graph 410 illustrates the structure prediction confidence for the 50 queries under each approach. In the first approach (plotted on the left), the average confidence across the 50 queries was ˜0.52, indicating so-so confidence in the structural predictions of the protein complexes. In the second approach (plotted on the right), the average confidence across the 50 queries was ˜0.73, indicating greatly improved confidence in the structural predictions of the protein complexes.

The second graph 420 illustrates inter-chain predicted aligned error (PAE) between residues of the target peptide and the targeting peptide in the structural configuration. Generally, lower predicted aligned errors indicate more confident structure predictions. Here, the first approach had an average PAE˜25.5, whereas the second approach had an average PAE˜25.

The third graph 430 illustrates subset root mean square deviation (subset RMSD) as a measure of agreement between a set of synthetic pairs and superimposed atomic coordinates from a predicted structure. Here, a lower subset RMSD indicates that targeting peptide residues and target peptide residues with variants identified as synthetic pairs are closer in space in a predicted structure. Here, the first approach (without synthetic augmentation) yielded an average RMSD˜21, whereas the second approach (with synthetic augmentation) yielded an average RMSD˜20.5. This indicates that the predicted structures for synergistic pairs generally had those pairs closer together than non-synergistic pairs, in agreement with the wet lab data used to identify the synergistic pairs.

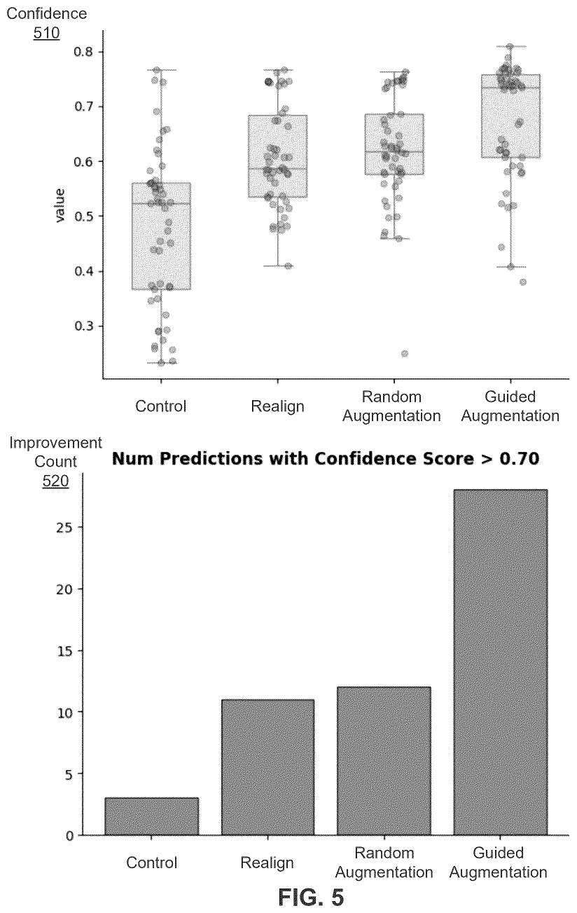

FIG. 5 illustrates additional graph results of synthetic augmentation of MSA compared to other non-augmented approaches, according to one or more example implementations.

The first approach (titled “Control”) refers to MSA prediction and structure prediction with just homolog pairs. The second approach (titled “Realign”) refers to MSA prediction and structure prediction with a superior MSA model (relative to the first approach). The third approach (titled “Random Augmentation”) refers to synthetic augmentation with random pairs of variants, without screening for synergistic pairs with high binding affinity. The fourth approach (titled “Guided Augmentation”) refers to synthetic augmentation with synthetic pairs identified as having high binding affinity. Of note, the fourth approach outperforms, not only the first approach without synthetic augmentation, but the MSA-improved approach and the random augmentation approach. In graph 510, the average confidence of the first approach is ˜0.5, the average confidence of the second approach and the third approach is ˜0.6, and the average confidence of the fourth approach is above 0.7. Moreover, the number of predictions (across the 50 queries) with an output confidence associated with the structural prediction of the protein complex above a common industry-wide threshold of 0.70 showed a significantly improved percentage via the guided augmentation approach.

### Example 2

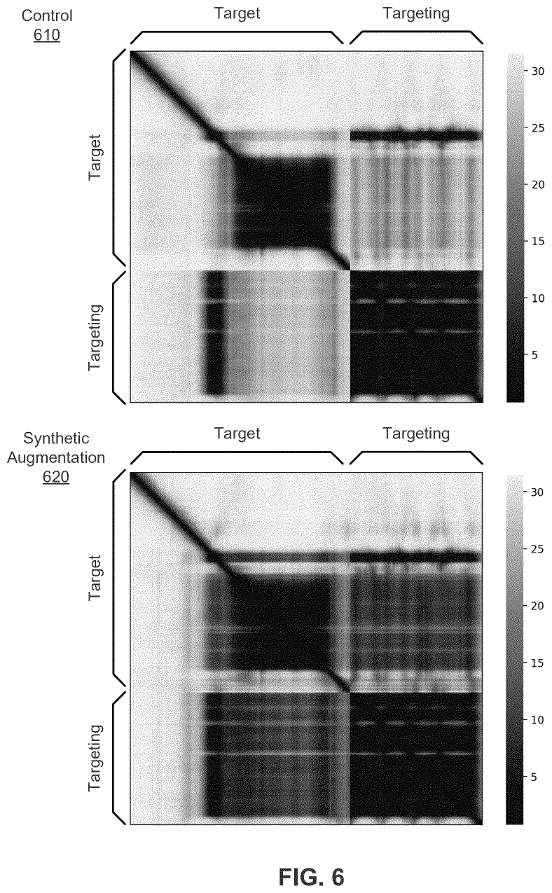

FIG. 6 illustrates predicted aligned error (PAE) of synthetic augmentation of MSA compared to a non-augmented approach, according to one or more example implementations. MSA and structure prediction was performed without synthetic augmentation (PAE plotted for Control 610) and with synthetic augmentation (PAE plotted for Synthetic Augmentation 620). The PAE along the diagonal was low for the Control 610, but had high PAE in the off-diagonal. Contrarily, the PAE of the Synthetic Augmentation 620 maintained low PAE in the diagonal, but improved relative to the Control 610 by lowering PAE in the off-diagonal as well. This further demonstrates the power and advantage in synthetic augmentation of MSA and structure prediction of protein complexes.

### Example 3

In order to understand protein-ligand interactions, such as protein-of-interest (POI) binding, single-site mutagenesis (SSM) was adopted. This approach involves systematically mutating individual amino acids within a protein to assess how each change affects binding. By analyzing the effects of these mutations, critical residues that contribute to the interaction between the protein and its binding partner can be identified.

A comprehensive dataset was generated by measuring binding affinities of a large number of variants of protein A against a large number of variants of protein B. The binding affinity was measured by the high-throughput screening system described in U.S. Pat. No. 11,466,265. Specifically, variants of protein A and variants of protein B were expressed on surfaces of two separate haploid strains of yeasts; and rates at which yeasts of two separate haploid strains fuse into diploids were measured. The rates represented binding affinities among various sets of protein A variants and protein B variants. This rich dataset enabled a detailed analysis of how modifications on either or both proteins influence their ability to bind to one another.

If a specific modification disrupts or enhances binding in a particular way-without compromising the overall structure or function of the protein-then the affected residue is likely located at the interface or plays a significant role in the structure of the interface. Because the experiment measured many pairs of variants, it was possible to infer which residues on the two proteins were functionally related. This allowed identification of interactions that are critical for binding specificity and affinity.

One particularly interesting phenotype observed in these experiments was the occurrence of synergistic mutation pairs. These are pairs of mutations that, when combined, exhibit behavior that is not simply the sum of their individual effects. The clearest example of this phenomenon is a compensatory effect: a mutation on protein A may disrupt binding to the wild-type (WT) protein B, and a mutation on protein B may disrupt binding to WT protein A, but when both mutations are present together, binding is restored—and in some cases, the affinity is even stronger than that of the original WT interaction.

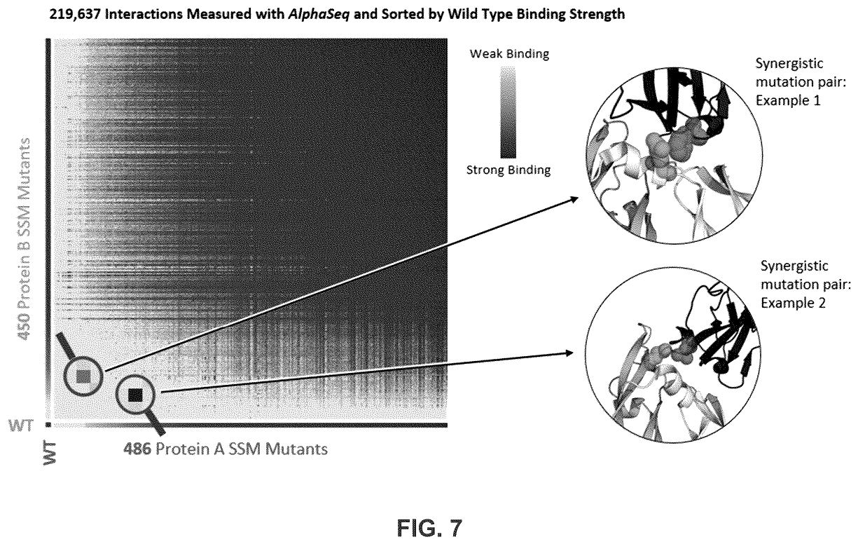

FIG. 7 visualizes these effects using a matrix of affinities for all possible mutation pairs. By sorting each mutation according to its binding to the wild-type partner, it became apparent that mutations which individually break binding (often represented by a yellow color in the matrix) can, in certain combinations, restore or even enhance binding. These distinct points in the matrix, where individual mutations had weak binding to WT but strong binding when paired, highlighted the presence of synergistic mutations.

The identification of these synergistic mutation pairs suggested which residues are located at the protein-protein interface. They also suggested the underlying mechanisms of binding and specificity, and allowed the design of proteins with altered or improved interaction properties.

To compute the synergistic effect of a mutation pair, the affinity of the double mutation was compared against the background distributions for each of the individual mutations. This approach yielded a normalized affinity score, which is particularly useful for identifying outliers-mutation pairs that exhibit nonlinear behavior when compared to the effects of the individual mutations alone. These outliers were identified where the combined effect of two mutations was significantly different from what would be expected if their effects were simply additive.

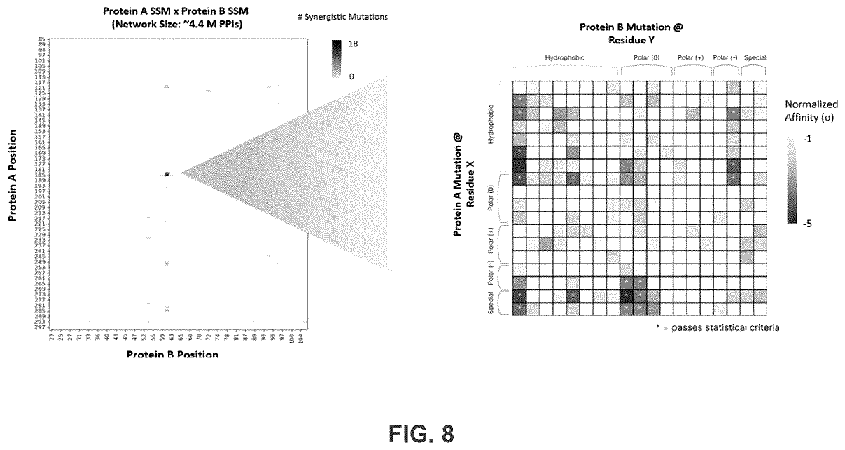

These effects were further analyzed at the residue level as provided in FIG. 8. For example, by zooming in on a 19 by 19 matrix that represents variant pairs at two specific residues, it was possible to examine systematically how each combination influences binding affinity. This matrix provided a comprehensive view of the interplay between mutations at these two positions, allowing us to observe patterns and identify pairs that have particularly strong or weak effects on binding.

Notably, some residue pairs displayed multiple synergistic mutations within this matrix. The presence of several such mutations suggested that these residues are tightly coupled, as their combined mutations consistently led to significant changes in binding affinity. This repeated evidence of synergy further supported that these residues may be in direct contact with each other or otherwise functionally linked within the protein structure.

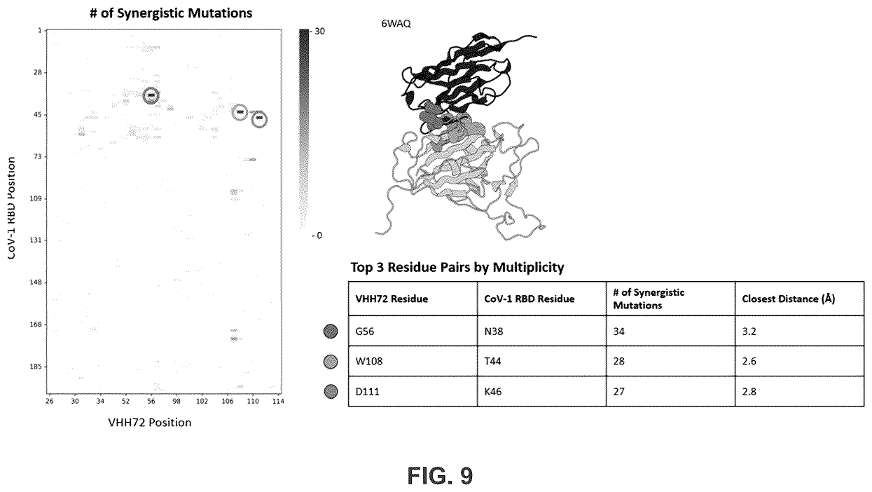

The synergistic mutations were also observed with VHH72 as illustrated in FIG. 9. On the left, a heatmap illustrates the number of synergistic mutations observed at each residue pair. The top three residue pairs by multiplicity are listed in the accompanying table on the right. This visual representation shows which residue pairs are most frequently involved in synergistic interactions.

When these highly synergistic residue pairs are mapped onto the known reference structure, these pairs are in close physical proximity to each other, validating the prediction. For example, as shown in FIG. 9, the two residues in red not only show a high degree of functional coupling through synergistic mutations but are also located near each other in the three-dimensional structure. This spatial closeness suggests a direct structural relationship that underlies their functional interaction.

These studies suggested that double-site saturation suppressor mutagenesis (dsSSMs) can be used to identify residue pairs that are functionally coupled. Importantly, the fact that these functionally coupled pairs are also structurally coupled suggested that they can serve as valuable constraints in computational modeling to improve the accuracy of complex structure prediction, as the identified residue pairs provide direct evidence of both functional and structural interdependence within the protein.

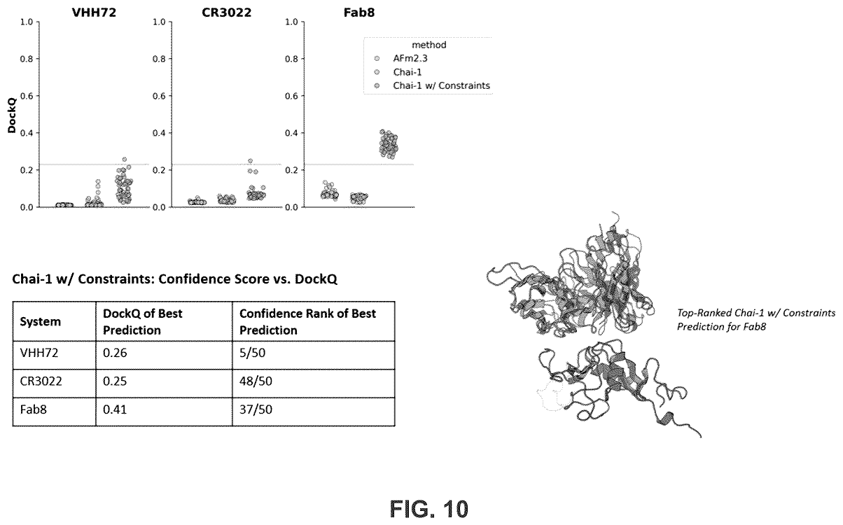

To test this, 50 samples was tested with Chai-1 both with and without constraints with the top three residue pairs identified as described above. Chai-1 is comparable to AlphaFold3 in that it is multimodal and leverages diffusion, but it also introduces the ability to provide constraints as input features. The results for VHH72, CR3022 and Fab8 are provided in FIG. 10. The results show that the addition of constraints led to an improvement. Particularly, all the results for Fab8 that included constraints surpassed the “acceptable quality” threshold, with the best prediction achieving a DockQ score of 0.41. Importantly, all of these predictions were physically viable, underscoring the practical value of integrating constraints.

### Example 4

The double-sided SSM data and the synergistic mutation pairs generated as described above were analyzed for the paired MSA features. This process allowed to create synthetic coevolution data that captured information about residues that are functionally and structurally coupled. These synergistic mutation pairs in the paired MSA provided AlphaFold with richer evolutionary signals and enabled to better predict the structure and interactions within the protein complex.

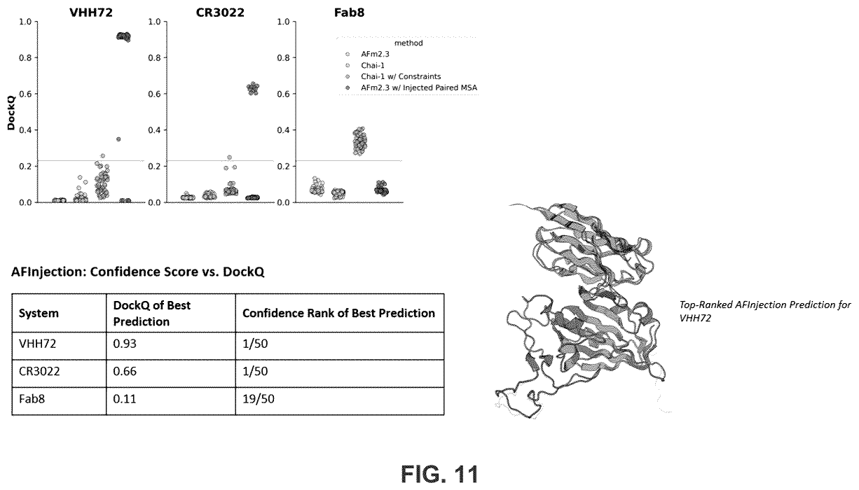

The results of the prediction are provided in FIG. 11. It shows a substantial improvement for both VHH72 and CR3022. The outcome for VHH72 was particularly significant-going from essentially zero to nearly perfect performance simply by modifying the MSA features. Specifically, for VHH72 a DockQ score reached 0.93, while for CR3022, the DockQ score reached 0.66.

The best predictions according to DockQ were top-ranked by predicted confidence. This means that even without the ground truth structures for these two pairs, the best models could be selected based on their predicted confidence scores.

To summarize, incorporating functional data as constraints significantly enhanced the ability to resolve complex prediction challenges without relying on extensive sampling. When the synergistic mutations were introduced into the paired multiple sequence alignment (MSA), it resulted in highly accurate predictions of the structure.

Predicting the structure of monomeric proteins has traditionally depended on the availability of extensive evolutionary information for individual protein sequences, typically obtained through multiple sequence alignments (MSAs) or protein language model (PLM) embeddings. In a similar vein, accurate prediction of protein complexes can require even richer and more comprehensive coevolutionary data.

In the study disclosed herein, high-throughput experimental assays offered a means to generate synthetic coevolutionary datasets. These datasets were particularly valuable for improving the accuracy of initial predictions of protein complex structures that may be suboptimal.

Beyond the practical benefits, these findings support that: high-throughput binding affinity data serves as a valuable and powerful resource for structural inference. The ability to leverage such data enabled to understand protein structure and function at a scale that was previously unattainable with conventional techniques.

### Additional Considerations

The foregoing description of the embodiments has been presented for the purpose of illustration; many modifications and variations are possible while remaining within the principles and teachings of the above description.

Any of the steps, operations, or processes described herein may be performed or implemented with one or more hardware or software modules, alone or in combination with other devices. In some embodiments, a software module is implemented with a computer program product comprising one or more computer-readable media storing computer program code or instructions, which can be executed by a computer processor for performing any or all of the steps, operations, or processes described. In some embodiments, a computer-readable medium comprises one or more computer-readable media that, individually or together, comprise instructions that, when executed by one or more processors, cause the one or more processors to perform, individually or together, the steps of the instructions stored on the one or more computer-readable media. Similarly, a processor may comprise one or more subprocessing units that, individually or together, perform the steps of instructions stored on a computer-readable medium.

Embodiments may also relate to a product that is produced by a computing process described herein. Such a product may store information resulting from a computing process, where the information is stored on a non-transitory, tangible computer-readable medium and may include any embodiment of a computer program product or other data combination described herein.

The description herein may describe processes and systems that use machine-learning models in the performance of their described functionalities. A “machine-learning model,” as used herein, comprises one or more machine-learning models that perform the described functionality. Machine-learning models may be stored on one or more computer-readable media with a set of weights. These weights are parameters used by the machine-learning model to transform input data received by the model into output data. The weights may be generated through a training process, whereby the machine-learning model is trained based on a set of training examples and labels associated with the training examples. The training process may include: applying the machine-learning model to a training example, comparing an output of the machine-learning model to the label associated with the training example, and updating weights associated for the machine-learning model through a back-propagation process. The weights may be stored on one or more computer-readable media, and are used by a system when applying the machine-learning model to new data.

The language used in the specification has been principally selected for readability and instructional purposes, and it may not have been selected to narrow the inventive subject matter. It is therefore intended that the scope of the patent rights be limited not by this detailed description, but rather by any claims that issue on an application based hereon.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive “or” and not to an exclusive “or”. For example, a condition “A or B” is satisfied by any one of the following: A is true (or present) and B is false (or not present); A is false (or not present) and B is true (or present); and both A and B are true (or present). Similarly, a condition “A, B, or C” is satisfied by any combination of A, B, and C being true (or present). As a not-limiting example, the condition “A, B, or C” is satisfied when A and B are true (or present) and C is false (or not present). Similarly, as another not-limiting example, the condition “A, B, or C” is satisfied when A is true (or present) and B and C are false (or not present). Further, “and/or” may refer to any combination of listed elements. For example, “A and/or B” is satisfied by A solely present, B solely present, or A and B present.

What is claimed is:

1. A method of generating a predicted structure of an interface between a target peptide and a targeting peptide, the method comprising:
obtaining sequences of (i) a first library of variants of the target peptide and (ii) a second library of variants of the targeting peptide;
generating a plurality of test pairs, wherein each test pair comprises one target variant selected from the variants of the target peptides and one targeting variant selected from the variants of the targeting peptide;
obtaining binding affinity data for each of the plurality of test pairs;
selecting one or more pairs out of the test pairs as one or more synergistic pairs based on the binding affinity data;
performing multiple sequence alignment with the sequences of target peptides and targeting peptides in the one or more synergistic pairs; and
generating the predicted structure of the interface between the target peptide and the targeting peptide based on the multiple sequence alignment.

2. The method of claim 1, wherein the one or more synergistic pairs are selected from test pairs for being a synergistic mutation pair.

3. The method of claim 1 or 2, wherein the one or more synergistic pairs are selected from test pairs having a combinative effect on binding affinity compared to individual effects of the target variant and the targeting variants above a threshold.

4. The method of claim 1 or 2, wherein the one or more synergistic pairs are selected from test pairs for having a binding affinity below a threshold.

5. The method of any one of claims 1-4, wherein the binding affinity data is obtained by a high-throughput analysis of binding between the test pairs.

6. The method of claim 5, wherein the high-throughput analysis is performed by a method comprising:
expressing variants of the target peptide in the first library and variants of the targeting peptide in the second library on surfaces of two separate haploid strains of yeasts; and
measuring rates at which yeasts of two separate haploid strains fuse into diploids, thereby obtaining the binding affinity data.

7. The method of claim 6, wherein the high-throughput analysis is performed in the presence of a mediating ligand.

8. The method of claim 7, wherein the binding affinity data indicate binding affinity among the variants of the target peptide, the variants of the targeting peptide, and the mediating ligand.

9. The method of claim 7 or 8, wherein the predicted structure of the interface is a structure of the interface in the presence of the mediating ligand between the target peptide and the targeting peptide.

10. The method of claim 9, wherein the predicted structure of the interface further comprises a structure of the mediating ligand.

11. The method of any one of claims 1-6, further comprising:
generating a structure of a mediating ligand or selecting a mediating ligand that can facilitate binding between the target peptide and the targeting peptide using the predicted structure of the interface between the target peptide and the targeting peptide.

12. The method of claim 11, further comprising:
producing the mediating ligand.

13. The method of claim 12, further comprising:
testing a binding affinity of the target peptide and the targeting peptide in the presence of the mediating ligand.

14. The method of any one of claims 9-13, further comprising:
modifying the mediating ligand or selecting an alternative ligand to improve binding to the target peptide and/or the targeting peptide.

15. The method of any one of claims 1-14, wherein the targeting peptide is an antibody, and the target peptide is an antigen.

16. The method of any one of claims 1-15, wherein the first library of variants of the target peptide comprises one or more homologs of the target peptide and the second library of variants of the targeting peptide comprises one or more homologs of the targeting peptide.

17. The method of claim 16, wherein the one or more homologs of the target peptide and/or the one or more homologs of the targeting peptide are generated by a generative machine-learning model.

18. The method of any one of claims 1-17, wherein the plurality of test pairs comprises one or more pairs of a homolog of the target peptide and a homolog of the targeting peptide.

19. The method of any one of claims 1-18, further comprising:
identifying a first set of amino acid residues of the targeting peptide contribute to binding to the target peptide based on the structure of the interface and a second set of amino acid residues of the targeting peptide that do not contribute to binding to the target peptide sequence.

20. The method of claim 19, further comprising:
generating an investigative variant of the targeting peptide by modifying one or more amino acid residues of the second set of amino acid residues of the targeting peptide sequence; and
receiving binding affinity data on the investigative variant of the targeting peptide and the target peptide.

21. The method of claim 20, further comprising:
determining that the binding affinity data on the investigative variant of the targeting peptide sequence and the target peptide sequence is greater than a threshold; and
producing the investigative variant of the targeting peptide.

22. The method of any one of claims 1-21, wherein performing the multiple sequence alignment with the sequences of target variants and targeting variants in the one or more synergistic pairs comprises:
performing sequence alignment of sequences of the targeting variants in the one or more synergistic pairs; and
performing sequence alignment of sequences of the target variants in the one or more synergistic pairs.

23. The method of any one of claims 1-22, further comprising:
identifying amino acid residues in the targeting peptide and amino acid residues in the target peptide that contribute to interaction between the targeting peptide and the target peptide based on the multiple sequence alignment,
wherein generating the predicted structure of the interface between the target peptide and the targeting peptide is further based on the identified amino acid residues in the targeting peptide and the identified amino acid residues in the target peptide that contribute to interaction between the targeting peptide and the target peptide.

24. The method of claim 23, wherein in the step of generating the predicted structure of the interface between the target peptide and the targeting peptide, the identified amino acid residues in the targeting peptide and the identified amino acid residues in the target peptide are used as constraints.

25. The method of any one of claims 1-24, wherein generating the predicted structure of the interface between the target peptide and the targeting peptide comprises:
applying a structure prediction model configured as a machine-learning model to the multiple sequence alignment to predict the structure of the interface.

26. The method of claim 25, wherein the structure prediction model is a machine-learning model developed using multiple sequence alignments of natural protein sequences for training, optionally wherein the natural protein sequences comprise natural homologs of the targeting peptide and/or the target peptide.

27. The method of claim 25 or claim 26, wherein the structure prediction model is configured to constrain structure prediction by establishing the amino acid residue residues in the targeting peptide and the amino acid residues in the target peptide as component to the binding interface of the targeting peptide and the target peptide.

28. The method of any one of claims 1-27, further comprising providing a confidence evaluation associated with the predicted structure of the interface between the target peptide and the targeting peptide, optionally wherein the confidence evaluation is represented by a PAE score.

29. The method of any one of claims 1-28, further comprising:
generating a digital representation of the structure of the binding interface between the target peptide and the targeting peptide.

30. The method of claim 29, generating a graphical user interface of the digital representation of the structure of the binding interface between the target peptide and the targeting peptide, wherein the graphical user interface is configured for display on a client device.

31. The method of claim 30, wherein generating the graphical user interface presenting the digital representation of the structure of the binding interface between the target peptide and the targeting peptide comprises:
tagging, in the digital representation, the amino acid residues in the targeting peptide sequence and the amino acid residues in the target peptide sequence that contribute to binding of the targeting peptide and the target peptide.

32. The method of any one of claims 1-31, wherein the first library of variants of the target peptide or the second library of variants of the targeting peptide comprises over 100 variants.

33. The method of any one of claim 1-32, wherein the plurality of test pairs comprises over 10,000 test pairs.

34. A non-transitory computer-readable storage medium storing instructions that, when executed by a computer processor, cause the computer processor to perform the method of any one of claims 1-33.

35. A system comprising:
a computer processor; and
the non-transitory computer-readable storage medium of claim 34.

36. A non-transitory computer-readable storage medium storing a predicted structure of an interface between a target peptide and a targeting peptide, the predicted structure generated by the method of any one of claims 1-33.

37. A graphical user interface for displaying a predicted structure of an interface between a target peptide and a targeting peptide, the predicted structure generated by the method of any one of claims 1-33.

38. A method of synthetic augmentation of multiple sequence alignment and structure prediction, the method comprising:
receiving, from a client device, a query including a target peptide sequence and a targeting peptide sequence;
querying a database to obtain one or more homolog pairs of the target peptide sequence and the targeting peptide sequence;
generating a plurality of variants of the target peptide sequence and a plurality of variants of the targeting peptide sequence;
transmitting the plurality of variants of the target peptide sequence and the plurality of variants of the targeting peptide sequence for binding affinity assaying;
receiving binding affinity data on each paired combination of one variant of the target peptide sequence and one variant of the targeting peptide sequence;
identifying one or more synergistic pairs, wherein each synergistic pair comprises one variant of the target peptide sequence and one variant of the targeting peptide sequence with binding affinity above a threshold;
performing multiple sequence alignment with the one or more homolog pairs and the one or more synergistic pairs; and
applying a structure prediction model to the multiple sequence alignment to predict a structure of a protein complex formed by the target peptide sequence and the targeting peptide sequence.
